(19) United States
(12) Patent Application Publication
(10) Pub. No.: US 2025/0271453 A1
(43) Pub. Date: Aug. 28, 2025

(54) PROTEIN ANTIGEN COMBINATION CONTAINING SERF2 AND APPLICATION THEREOF

(71) Applicant: SHANGHAI ZHONGQI BIOTECHNOLOGY CO., LTD., Shanghai (CN)

(72) Inventor: Liqin CAO, Shanghai (CN)

(21) Appl. No.: 19/206,017

(22) Filed: May 12, 2025

Related U.S. Application Data
(63) Continuation of application No. PCT/CN2023/134613, filed on Nov. 28, 2023.

(30) Foreign Application Priority Data
Nov. 29, 2022 (CN) 202211507225.7

(51) Int. Cl. G01N 33/68

(52) U.S. Cl. CPC G01N 33/6896; G01N 2800/2821

(57) ABSTRACT

A protein antigen combination containing SERF2 and applications thereof in the field of biological detection are disclosed. The antigen combination for detecting autoantibodies can distinguish Alzheimer's disease (AD) from frontotemporal dementia (FTD) and dementia with Lewy bodies (DLB), and the antigen combination at least includes protein fragments of SERF2. The new protein antigen composition can not only effectively identify patients with AD, but also effectively distinguish AD from FTD and DLB, enabling accurate identification of AD. It is of great importance in terms of diagnostic applications and research.

### CROSS-REFERENCE TO RELATED APPLICATIONS

This application is a continuation of International Pat. Appl. No. PCT/CN2023/134613, filed on Nov. 28, 2023, which claims priority to Chinese Pat. Appl. No. 202211507225.7, filed on Nov. 29, 2022, the contents of each of which are incorporated by reference herein in their entireties.

### REFERENCE TO AN ELECTRONIC SEQUENCE LISTING

The contents of the electronic sequence listing (SHCH-2200791US.xml; Size: 12,288 bytes; and Date of Creation: May 13, 2025) is incorporated herein by reference in its entirety.

### TECHNICAL FIELD

The present disclosure belongs to the field of biological detection, and particularly relates to a protein antigen combination containing SERF2 for detecting autoantibodies that can distinguish Alzheimer's disease from frontotemporal dementia and dementia with Lewy bodies, and applications thereof.

### BACKGROUND

Alzheimer's disease (AD for short) is a progressive neurodegenerative disease mainly characterized by memory and cognitive disorder. It frequently occurs in the elderly, and the course of disease is slow and irreversible. AD is the most common dementia type, accounting for 60%-70% of all patients with dementia. Existing diagnostic methods for AD primarily make a judgment based on a Mini-Mental State Examination (MMSE) score and one or more imaging tests, such as magnetic resonance imaging (MRI), computerized tomography (CT) and/or positron emission tomography (PET), in combination with the patient's clinical symptoms.

Autoantibodies are antibodies produced by an individual's immune system against the individual's own protein antigens. Under normal circumstances, the immune system produces antibodies in response to exogenous proteins or substances in the bodies. However, the immune system sometimes also recognizes one or more endogenous components in the bodies, resulting in the production of autoantibodies. There has been extensive evidence to confirm that a plurality of autoantibodies in serum are involved in neurological diseases and syndromes. Chinese patent CN 110850104 B discloses a series of antigen proteins and/or fragments and combinations thereof, capable of being used for autoantibody detection of patients with AD. These combinations can have a good effect when used for diagnosing AD clinically, but have not been involved in distinguishing and identifying AD, DLB, FTD and other relevant dementia.

DLB is one of the most common neurodegenerative diseases, and its main pathological feature is widely distributed Lewy bodies (LBs) in cerebral cortices and brain stems of patients. According to data statistics, the prevalence of DLB in elderly people aged 65 years or above is 3.6%-6.6%, accounting for 10%-20% of patients with dementia. Many clinical symptoms and pathological manifestations of DLB are similar to those of AD, and LBs can be found in brains of 40% or more of patients with AD, which proves a high overlap between AD and DLB. In addition, in clinical manifestations, both DLB and AD show progressive cognitive disorder, and they were indistinguishable in severity. Contents of Tau proteins and beta amyloid proteins in cerebrospinal fluid are typically detected, both of which show similar changes in both diseases. Thus, the two diseases are difficult to distinguish and diagnose clinically. At present, the two diseases are mainly distinguished and identified clinically through imaging examinations. One method performs a cranial MRI examination to find a tiny difference between the two diseases in certain brain atrophy areas, and another method performs cerebral perfusion SPECT/PET examination to distinguish AD from DLB through imaging changes. The existing distinguishing methods have high requirements for the physician's experience.

FTD is a progressive neurodegenerative disease with hidden onset. It is a dementia syndrome characterized by frontal or frontotemporal atrophy, accounting for about 5%-15% of all dementia types. Clinically, patients with FTD and patients with AD develop cognitive disorder, behavior disorder and language disorder to different degrees, and MMSE scores of the patients with FTD do not obviously differ from those of the patients with AD. At present, CT and MRI are typically used for identifying FTD and AD. Generalized cerebral atrophy is visible in the patients with AD, and frontal and/or temporal lobe atrophy is visible in frontotemporal dementia. However, further histopathological examination is still generally required to confirm the diagnosis.

It is impossible to distinguish the above diseases by biomarkers in existing studies. Paterson et al. (Alzheimer's Research & Therapy (2018) 10:32) detected contents of ten kinds of Tau proteins, different fragments of beta amyloid proteins (A beta), precursor proteins (APP), NFL, YKL-40 and the like in cerebrospinal fluid of patients with a variety of dementias and proportions of relevant protein contents, and the results proved that these markers cannot effectively identify and distinguish AD from DLB. A beta 42/40 has certain reference significance in identifying and distinguishing AD from behavioral variant frontotemporal dementia (bvFTD). However, it has the shortcoming that a certain risk is brought to patients by requiring a cerebrospinal fluid sample, in that the patients usually reject it.

Meanwhile, in contrast with autopsy results, the clinical diagnosis of AD still has a misdiagnosis rate of 25%-30%, even in professional dementia diagnosis and treatment centers. AD is a progressive neurodegenerative disease, its symptoms are gradually aggravated as the course is prolonged, its course is long, and it progresses slowly in the early period of onset. Thus, the earlier the diagnosis and intervention is made, the easier the disease is controlled, and the more the patients can benefit. However, the detection accuracy of instruments such as MRI and PET is lower in early stages of the diseases, due to the small or insignificant pathological changes and/or differences. Moreover, clinical symptoms of AD, FTD, DLB and other dementias are similar in the early periods of the diseases, which further increases the difficulty in clinical diagnosis.

Thus, it is of great practical significance to develop a diagnosis technique and kit capable of effectively identifying AD and distinguishing AD identifying AD from FTD, DLB and other dementias.

### SUMMARY

The present disclosure aims at providing a protein antigen combination for detecting autoantibodies that can effectively identify AD and distinguish AD from FTD, DLB and other dementia, and applications thereof.

In order to achieve the objectives of the present disclosure, the technical solution adopted in the present disclosure provides an antigen combination that includes at least a SERF2 protein and/or a SNAP25 protein.

Preferably, the SERF2 protein includes the amino acid sequence shown in SEQ ID NO:7, and the SNAP25 protein includes the amino acid sequence shown in SEQ ID NO:8.

Preferably, the antigen combination includes both the SERF2 protein and the SNAP25 protein.

Preferably, the antigen combination further includes one or more of a MAPT protein fragment, a RAGE protein fragment, an ASXL1 protein fragment, a JMJD2D protein fragment, a P21 protein fragment and a DNAJC8 protein fragment.

Preferably, the MAPT protein fragment includes the amino acid sequence shown in SEQ ID NO:1, the RAGE protein fragment includes the amino acid sequence shown in SEQ ID NO:4, the ASXL1 protein fragment includes the amino acid sequence shown in SEQ ID NO:5, the JMJD2D protein fragment includes the amino acid sequence shown in SEQ ID NO:6, the P21 protein fragment includes the amino acid sequence shown in SEQ ID NO:2, and the DNAJC8 protein fragment includes the amino acid sequence shown in SEQ ID NO:3.

Accordingly, the antigen combination can be applied in preparation of products for detecting/identifying Alzheimer's disease.

Accordingly, the antigen combination can be applied in preparation of products for distinguishing Alzheimer's disease from frontotemporal dementia and dementia with Lewy bodies.

Accordingly, a kit that detects and/or identifies Alzheimer's disease and that preferably distinguishes Alzheimer's disease from frontotemporal dementia and dementia with Lewy bodies can include or be prepared from the antigen combination.

Accordingly, a SERF2 protein, a SNAP25 protein, a MAPT protein fragment, a RAGE protein fragment, an ASXL1 protein fragment, a JMJD2D protein fragment, a P21 protein fragment and/or a DNAJC8 protein fragment can be applied in preparation of products for detecting and/or identifying Alzheimer's disease, wherein the SERF2 protein has the amino acid sequence shown in SEQ ID NO:7, the SNAP25 protein has the amino acid sequence shown in SEQ ID NO:8, the MAPT protein fragment has the amino acid sequence shown in SEQ ID NO:1, the RAGE protein fragment has the amino acid sequence shown in SEQ ID NO:4, the ASXL1 protein fragment has the amino acid sequence shown in SEQ ID NO:5, the JMJD2D protein fragment has the amino acid sequence shown in SEQ ID NO:6, the P21 protein fragment has the amino acid sequence shown in SEQ ID NO:2, and the DNAJC8 protein fragment has the amino acid sequence shown in SEQ ID NO:3.

Accordingly, a SERF2 protein, a SNAP25 protein, a MAPT protein fragment, a RAGE protein fragment, an ASXL1 protein fragment, a JMJD2D protein fragment, a P21 protein fragment and/or a DNAJC8 protein fragment can be applied in preparation of products for distinguishing Alzheimer's disease from frontotemporal dementia and dementia with Lewy bodies, wherein the SERF2 protein has the amino acid sequence shown in SEQ ID NO:7, the SNAP25 protein has the amino acid sequence shown in SEQ ID NO:8, the MAPT protein fragment has the amino acid sequence shown in SEQ ID NO:1, the RAGE protein fragment has the amino acid sequence shown in SEQ ID NO:4, the ASXL1 protein fragment has the amino acid sequence shown in SEQ ID NO:5, the JMJD2D protein fragment has the amino acid sequence shown in SEQ ID NO:6, the P21 protein fragment has the amino acid sequence shown in SEQ ID NO:2, and the DNAJC8 protein fragment has the amino acid sequence shown in SEQ ID NO:3.

The present disclosure has the following beneficial effects. The present disclosure provides a new protein antigen composition that can not only effectively identify patients with AD, but can also effectively distinguish AD from FTD and DLB, enabling accurate identification of AD. It is of great importance in applications and research in diagnosing and treating various forms of dementia.

### DETAILED DESCRIPTION OF THE EMBODIMENTS

The present application will be further described below with reference to specific examples. Unless otherwise specified, experimental methods used in the following examples are conventional methods, and materials, reagents and the like used in the examples are available commercially. Data obtained are average values from at least three repetitions, and the data obtained from each repetition is valid data.

### Example 1: Construction, Expression and Purification of Recombinant Vector of Protein Antigen

1. Selection of antigen. 23 antigen proteins highly related to Alzheimer's disease were selected for construction, expression and purification. Respective database IDs of the antigen proteins are shown in Table 1.

TABLE 1
Database ID correspondence table of proteins to be tested

| Protein name | Database ID | Protein name | Database ID |
|---|---|---|---|
| MAPT | NP_058519.3 | ICAM1 | NP_000192.2 |
| ADARB1 | NP_001103.1 | CHGA | NP_001266.1 |
| P21 | NP_000380.1 | SNAP25 | NP_001309832.1 |
| DNAJC8 | NP_055095.2 | VSNL1 | NP_001353732.1 |
| RAGE | NP_001127.1 | CHI3L1 | NP_001267.2 |
| ASXL1 | NP_056153.2 | FABP3 | NP_001307925.1 |
| JMJD2D | NP_060509.2 | AD7c-NTP | AAC08737.1 |
| ApoE4 | NP_001289617.1 | RALGPS2 | NP 689876.2 |
| H2BC5 | NP_066407.1 | CTSH | NP_004381.2 |
| TOMM20 | NP_055580.1 | DOC2A | NP_003577.2 |
| PDPN | NP_006465.3 | ICA1L | NP_001275551.1 |
| SERF2 | NP_001018118.1 | — | — |

2. Construction and expression of recombinant vectors for antigens. Using a human cDNA library (purchased from Invitrogen/Thermo Fisher Scientific Inc.) or whole gene synthetic DNA as a template, primers were respectively designed, and a full-length gene of the antigen protein was cloned into a pET28 plasmid by PCR, enzyme digestion, ligation and other molecular cloning methods. Meanwhile, HIS, c-myc and other tags were added to the N-terminal of the antigen protein to form a fusion protein. The recombinant expression vector was identified by DNA sequencing and confirmed to contain a correct antigen protein gene fragment. It should be noted that the added tag(s) is merely convenient for identification and extraction of the protein, and does not have a decisive impact on the antigenic function(s) of the protein. When in use, the tag may not necessarily be added, or other tags may be added as needed or desired.

The recombinant plasmids containing the protein gene fragments in Table 1 were individually transformed into competent cells of E. coli BL21 (DE3). The clones were selected and inoculated into LB media, and then shake-cultured at 37° C. When the E. coli BL21 (DE3) density OD600 reached approximately 0.8, the temperature was reduced to 16° C., 0.1 mM isopropylthio-beta-D-galactoside (IPTG) was added to each LB medium to induce expression overnight, and bacterial cells were obtained.

3. Purification of antigen. The expression-induced bacterial cells were collected by centrifugation and rinsed twice with phosphate-buffered saline (PBS). The bacterial cells were resuspended and dispersed in a lysis buffer (5-10 mL of lysis buffer per gram of bacterial cells), and were then subjected to ultrasonic disruption (ultrasound power: 200 W, ultrasonication for 5 seconds with a 5-second interval) in an ice bath. After cell disruption, the bacterial cells were centrifuged at 13000 rpm and at 10° C. for 20 minutes. A supernatant was collected, and then purified via two steps: Ni column affinity chromatography and molecular sieve chromatography. The purified target protein was analyzed by sodium dodecyl sulfate polyacrylamide gel electrophoresis (SDS-PAGE) to confirm the molecular weight and purity of the protein, and after the concentration of the protein was determined via the Bradford method, the protein was stored at −80° C. for later use. The purified proteins to be tested were thus obtained.

### Example 2: Candidate Antigens were Screened from Proteins to be Tested

1. Solutions and reagents used in this example were as follows:

(1) The coating buffer was a PBS buffer (pH=7.4). The preparation method was as follows: 3.58 g of Na₂HPO₄·12H₂O, 0.23 g of KH₂PO₄·2H₂O, 0.2 g of KCl and 8.0 g of NaCl were accurately weighed and dissolved in deionized (DI) water, and then diluted with DI water to 1 L.

(2) Blocking buffer/sample diluent/antibody diluent: 10 g of BSA (bovine serum albumin) was dissolved in the coating buffer, and diluted with DI water to 1 L.

(3) Washing buffer: The washing buffer was prepared by adding 0.5% Tween 20 (V/V) to the coating buffer before use (pH=7.4).

(4) 3,3′,5,5′-Tetramethylbenzidine (TMB) chromogenic agent was purchased from KPL.

(5) Stopping solution: 1 M hydrochloric acid.

2. The protein to be tested was coated onto a solid phase support. Each purified protein in Example 1 to be tested was diluted to a concentration of 5 g/ml using the coating buffer, and the diluted protein was then added to a 96-well plate at 50 L/well and coated overnight at 4° C. for incubation. On the following day, the solution was discarded, and the plate was air-dried and washed with the washing buffer three times, with each well receiving 200 L of washing buffer per time. 200 L of blocking buffer was added to each well and incubated at room temperature for 1 hour, and then the plate was air-dried after the blocking buffer was discarded, washed with the washing buffer three times (each well receiving 200 L of washing buffer each time), and air-dried again to obtain a solid phase-coated protein antigen in the 96-well plate.

3. The sample to be tested was added. Human serum to be tested was diluted by a factor of 100 with the sample diluent and then added to the 96-well plate containing the protein to be tested. 50 μL of the diluted sample to be tested was added to each well. The 96-well plate was then placed on a microplate shaker, where it was incubated at room temperature for 1 hour. The plate was then air-dried, washed with the washing buffer three times (each well receiving 200 L of washing buffer each time), and then air-dried again.

4. An enzyme-labeled second antibody was added. 1.0 mg/mL horseradish peroxidase-labeled recombinant goat anti-human immunoglobulin G antibody (purchased from Jackson Immune Research Inc.) was diluted by a factor of 20,000 with the antibody diluent, and then added to the 96-well plate from step 3 in a volume of 50 L/well. The 96-well plate was then placed on a microplate shaker, where it was incubated at room temperature for 0.5 hours. The plate was air-dried, washed with the washing buffer three times (each well receiving 200 L of washing buffer each time), and then air-dried again.

5. Chromogenic reaction and optical density reading. In the 96-well plate from step 4, 50 L/well of the TMB chromogenic agent was added to the plate, followed by shaking for 15 seconds. The reaction was kept away from light for 15 minutes at room temperature, and then 50 L of stopping solution was added. The absorbance at 450 nm was read using a microplate reader to obtain a detection signal (S) of each sample to be tested.

6. Analysis of sensitivity and specificity. 180 positive samples (sera of patients diagnosed with Alzheimer's Disease) and 180 negative samples (sera of healthy subjects) were taken, and the detection signal (S) of each sample was measured according to the above method (absorption value at a light wavelength of 450 nm). The negative samples were taken as negative reference samples. The mean (M, average value) and standard deviation (SD) of the detection signals (S) of all of the negative reference samples were calculated, and M+3*SD was taken as a cut-off value. The samples (S≥M+3*SD) with a detection signal (S)≥the cut off value were regarded as positive, and the samples (S<M+3*SD) with a detection signal (S)<the cut off value were regarded as negative.

The specificity and sensitivity were calculated based on the positive and negative results of the samples. The specificity refers to the proportion of healthy subjects whose samples are correctly determined as negative; that is, the number of the negative samples that are correctly determined as negative divided by the total number of the negative samples. The sensitivity refers to the proportion of samples of patients with Alzheimer's disease that are determined as positive; that is, the number of the positive samples that are correctly determined as positive divided by the total number of the positive samples. The sensitivity and specificity of each protein to be tested as an antigen for sample detection are calculated, and results are shown in Table 2.

TABLE 2
Sensitivity and specificity of each protein
to be tested/protein fragment as antigen

| | Translation start site and | | |
|---|---|---|---|
| | translation termination | | |
| Test antigen | site of amino acid | Sensitivity | Specificity |
| MAPT | 502-758 | 40.56% | 90.00% |
| P21 | 2-164 | 37.78% | 90.56% |
| DNAJC8 | 114-253 | 35.00% | 88.89% |
| RAGE | 23-54 | 34.44% | 89.44% |
| ASXL1 | 1-84 | 33.89% | 90.00% |
| JMJD2D | 1-160 | 33.33% | 89.44% |
| CHI3L1 | 21-383 | 2.22% | 97.92% |
| ApoE4 | 19-317 | 3.89% | 98.33% |
| | 19-128 | 2.22% | 99.44% |
| | 82-207 | 4.44% | 99.44% |
| SERF2 | 1-59 | 25.56% | 95.56% |
| ICAM1 | 27-532 | 6.11% | 96.67% |
| | 28-480 | 5.56% | 90.56% |
| | 504-532 | 8.33% | 90.56% |
| CHGA | 18-457 | 3.89% | 93.33% |
| SNAP25 | 1-206 | 28.89% | 95.56% |
| RALGPS2 | 1-583 | 8.33% | 96.11% |
| | 1-194 | 2.78% | 95.56% |
| | 163-358 | 5.00% | 100.00% |
| | 342-583 | 5.56% | 97.78% |
| H2BC5 | 1-126 | 4.44% | 100.00% |
| | 78-126 | 8.33% | 99.44% |
| TOMM20 | 1-145 | 2.78% | 97.78% |
| | 72-145 | 9.44% | 95.56% |
| PDPN | 23-162 | 2.78% | 97.92% |
| | 23-127 | 5.56% | 93.33% |
| ICA1L | 1-482 | 2.22% | 90.56% |
| | 1-202 | 1.67% | 93.89% |
| | 151-385 | 4.44% | 89.44% |
| | 261-482 | 3.89% | 90.00% |
| AD7c-NTP | 1-375 | 15.56% | 76.11% |
| CTSH | 22-335 | 5.56% | 93.33% |
| DOC2A | 1-400 | 6.11% | 90.56% |
| | 1-89 | 4.44% | 97.92% |
| | 76-211 | 8.33% | 95.56% |
| | 215-400 | 2.22% | 97.78% |
| | 251-384 | 9.44% | 94.44% |
| FABP3 | 1-133 | 2.78% | 95.56% |
| VSNL1 | 1-191 | 7.78% | 98.33% |

7. Proteins with sensitivity greater than or equal to 15% and specificity greater than or equal to 85% were selected and screened from the proteins or fragments above to be tested as candidate antigens. Screening results are shown in Table 3.

TABLE 3
Candidate antigens screened from proteins to be tested

| | Translation start | | | Corres- |
|---|---|---|---|---|
| | site and translation | | | ponding |
| | termination site | Sensi- | Speci- | amino acid |
| Test antigen | of amino acid | tivity | ficity | sequence |
| MAPT | 502-758 | 40.56% | 90.00% | SEQ ID NO: 1 |
| P21 | 2-164 | 37.78% | 90.56% | SEQ ID NO: 2 |
| DNAJC8 | 114-253 | 35.00% | 88.89% | SEQ ID NO: 3 |
| RAGE | 23-54 | 34.44% | 89.44% | SEQ ID NO: 4 |
| ASXL1 | 1-84 | 33.89% | 90.00% | SEQ ID NO: 5 |
| JMJD2D | 1-160 | 33.33% | 89.44% | SEQ ID NO: 6 |
| SERF2 (Full- | 1-59 | 25.56% | 95.56% | SEQ ID NO: 7 |
| length protein) | | | | |
| SNAP25 (Full- | 1-206 | 28.89% | 95.56% | SEQ ID NO: 8 |
| length protein) | | | | |

### Example 3: Display of the Effectiveness of Candidate Antigens for Detecting Samples of Patients with DLB and FTD

43 DLB positive samples (serum of patients diagnosed with DLB), 63 FTD positive samples (serum of patients diagnosed with FTD) and 232 negative samples (serum of healthy subjects) were taken respectively, and a detection signal (S) of each sample was measured using the candidate antigens (protein fragments) in Table 3 according to the method in Example 2. The negative samples were taken as negative reference samples. The mean (M, average value) and standard deviation (SD) of detection signals (S) of all negative reference samples were calculated, and M+3*SD was taken as a cut-off value. The samples (S≥M+3*SD) with a detection signal (S)≥the cut off value were regarded as positive, and the samples (S<M+3*SD) with a detection signal (S)<cut off value were regarded as negative.

The specificity and sensitivity were calculated based on the positive and negative results of the samples. The specificity refers to the proportion of healthy subjects whose samples are correctly determined as negative; that is, the number of the negative samples which are correctly determined as negative divided by the total number of the negative samples. The sensitivity of DLB samples refers to the proportion of samples from DLB patients with DLB that are correctly determined as positive; that is, the number of the positive samples in the DLB positive samples divided by the total number of the positive samples. The sensitivity of FTD samples refers to the proportion of samples from patients with FTD that are correctly determined as positive; that is, the number of the positive samples in the FTD positive samples divided by the total number of the positive samples. The sensitivity and specificity of each protein to be tested as an antigen for sample detection are calculated, and results are shown in Table 4.

TABLE 4
Test results of samples of patients with DLB and FTD

| | Translation start | | | |
|---|---|---|---|---|
| | and termination | | | |
| Test | sites of amino | Sensitivity | Sensitivity | |
| antigen | acid sequence | for DLB | for FTD | Specificity |
| MAPT | 502-758 | 4.65% | 19.05% | 90.09% |
| P21 | 2-164 | 0% | 1.59% | 90.52% |
| DNAJC8 | 114-253 | 0% | 4.76% | 89.66% |
| RAGE | 23-54 | 9.30% | 1.59% | 90.52% |
| ASXL1 | 1-84 | 16.28% | 4.76% | 90.95% |
| JMJD2D | 1-160 | 4.65% | 7.94% | 90.09% |
| SERF2 | 1-59 | 4.65% | 4.76% | 95.69% |
| SNAP25 | 1-206 | 4.65% | 6.35% | 95.26% |

### Example 4: Display of the Effectiveness of Candidate Antigen Combinations for Detecting AD, FTD and DLB

1. Proteins or protein fragments were selected from the candidate protein antigens in Table 3 to form different antigen combinations, each containing SERF2 and SNAP25. The specific combinations are shown in Table 5.

TABLE 5
Antigen combinations

| Combination | Antigens |
|---|---|
| Combination 1 | SERF2, SNAP25 |
| Combination 2 | SERF2, SNAP25, MAPT |
| Combination 3 | SERF2, SNAP25, DNAJC8 |
| Combination 4 | SERF2, SNAP25, P21 |
| Combination 5 | SERF2, SNAP25, ASXL1 |
| Combination 6 | SERF2, SNAP25, JMJD2D |
| Combination 7 | SERF2, SNAP25, RAGE |
| Combination 8 | SERF2, SNAP25, MAPT, DNAJC8 |
| Combination 9 | SERF2, SNAP25, MAPT, P21 |
| Combination 10 | SERF2, SNAP25, MAPT, ASXL1 |
| Combination 11 | SERF2, SNAP25, MAPT, JMJD2D |
| Combination 12 | SERF2, SNAP25, MAPT, RAGE |
| Combination 13 | SERF2, SNAP25, DNAJC8, P21 |
| Combination 14 | SERF2, SNAP25, DNAJC8, ASXL1 |
| Combination 15 | SERF2, SNAP25, DNAJC8, JMJD2D |
| Combination 16 | SERF2, SNAP25, DNAJC8, RAGE |
| Combination 17 | SERF2, SNAP25, P21, ASXL1 |
| Combination 18 | SERF2, SNAP25, P21, JMJD2D |
| Combination 19 | SERF2, SNAP25, P21, RAGE |
| Combination 20 | SERF2, SNAP25, ASXL1, JMJD2D |
| Combination 21 | SERF2, SNAP25, ASXL1, RAGE |
| Combination 22 | SERF2, SNAP25, JMJD2D, RAGE |
| Combination 23 | SERF2, SNAP25, MAPT, DNAJC8, P21 |
| Combination 24 | SERF2, SNAP25, MAPT, DNAJC8, ASXL1 |
| Combination 25 | SERF2, SNAP25, MAPT, DNAJC8, JMJD2D |
| Combination 26 | SERF2, SNAP25, MAPT, DNAJC8, RAGE |
| Combination 27 | SERF2, SNAP25, MAPT, P21, ASXL1 |
| Combination 28 | SERF2, SNAP25, MAPT, P21, JMJD2D |
| Combination 29 | SERF2, SNAP25, MAPT, P21, RAGE |
| Combination 30 | SERF2, SNAP25, MAPT, ASXL1, JMJD2D |
| Combination 31 | SERF2, SNAP25, MAPT, ASXL1, RAGE |
| Combination 32 | SERF2, SNAP25, MAPT, JMJD2D, RAGE |
| Combination 33 | SERF2, SNAP25, DNAJC8, P21, ASXL1 |
| Combination 34 | SERF2, SNAP25, DNAJC8, P21, JMJD2D |
| Combination 35 | SERF2, SNAP25, DNAJC8, P21, RAGE |
| Combination 36 | SERF2, SNAP25, DNAJC8, ASXL1, JMJD2D |
| Combination 37 | SERF2, SNAP25, DNAJC8, ASXL1, RAGE |
| Combination 38 | SERF2, SNAP25, DNAJC8, JMJD2D, RAGE |
| Combination 39 | SERF2, SNAP25, P21, ASXL1, JMJD2D |
| Combination 40 | SERF2, SNAP25, P21, ASXL1, RAGE |
| Combination 41 | SERF2, SNAP25, P21, JMJD2D, RAGE |
| Combination 42 | SERF2, SNAP25, ASXL1, JMJD2D, RAGE |
| Combination 43 | SERF2, SNAP25, MAPT, DNAJC8, P21, ASXL1 |
| Combination 44 | SERF2, SNAP25, MAPT, DNAJC8, P21, JMJD2D |
| Combination 45 | SERF2, SNAP25, MAPT, DNAJC8, P21, RAGE |
| Combination 46 | SERF2, SNAP25, MAPT, DNAJC8, ASXL1, |
| | JMJD2D |
| Combination 47 | SERF2, SNAP25, MAPT, DNAJC8, ASXL1, RAGE |
| Combination 48 | SERF2, SNAP25, MAPT, DNAJC8, JMJD2D, RAGE |
| Combination 49 | SERF2, SNAP25, MAPT, P21, ASXL1, JMJD2D |
| Combination 50 | SERF2, SNAP25, MAPT, P21, ASXL1, RAGE |
| Combination 51 | SERF2, SNAP25, MAPT, P21, JMJD2D, RAGE |
| Combination 52 | SERF2, SNAP25, MAPT, ASXL1, JMJD2D, RAGE |
| Combination 53 | SERF2, SNAP25, DNAJC8, P21, ASXL1, JMJD2D |
| Combination 54 | SERF2, SNAP25, DNAJC8, P21, ASXL1, RAGE |
| Combination 55 | SERF2, SNAP25, DNAJC8, P21, JMJD2D, RAGE |
| Combination 56 | SERF2, SNAP25, DNAJC8, ASXL1, JMJD2D, |
| | RAGE |
| Combination 57 | SERF2, SNAP25, P21, ASXL1, JMJD2D, RAGE |
| Combination 58 | SERF2, SNAP25, MAPT, DNAJC8, P21, ASXL1, |
| | JMJD2D |
| Combination 59 | SERF2, SNAP25, MAPT, DNAJC8, P21, ASXL1, |
| | RAGE |
| Combination 60 | SERF2, SNAP25, MAPT, DNAJC8, P21, |
| | JMJD2D, RAGE |
| Combination 61 | SERF2, SNAP25, MAPT, DNAJC8, ASXL1, |
| | JMJD2D, RAGE |
| Combination 62 | SERF2, SNAP25, MAPT, P21, ASXL1, JMJD2D, |
| | RAGE |
| Combination 63 | SERF2, SNAP25, DNAJC8, P21, ASXL1, |
| | JMJD2D, RAGE |
| Combination 64 | SERF2, SNAP25, MAPT, DNAJC8, P21, ASXL1, |
| | JMJD2D, RAGE |

2. 94, 63 and 44 serum samples of patients with AD, FTD and DLB were taken respectively, and 197 serum samples of normal healthy subjects were taken. Each sample was tested for sensitivity and specificity using the detection method in Example 2 for each antigen combination in Table 5.

The methods of Example 2 and Example 3 were used to define whether each antigen in the antigen combination is positive or negative. The method for defining overall sensitivity and specificity of the antigen combination is as follows:

For the antigen combinations, when the detection signal obtained from a certain serum sample using any antigen in the combination is a positive detection signal, the serum sample is a positive sample. Otherwise, the serum sample is a negative sample. Based on the positive and negative definitions above, positive and negative results were obtained from the samples using a given antigen combination, and then the sensitivity of the given antigen combination for the patient samples was calculated.

The sensitivity refers to the proportion of samples of AD (or FTD or DLB) patients that are determined as positive; that is, the number of the positive samples in all the samples of the AD (or FTD or DLB) patients divided by the total number of all the samples of the AD (or FTD or DLB) patients. The specificity refers to the proportion of healthy subjects whose samples are correctly determined as negative; that is, the number of the negative samples which are correctly determined as negative divided by the total number of the negative samples.

Sensitivity and specificity detected and calculated for each antigen combination are shown in Table 6.

TABLE 6
Detection results for each antigen combination

| | Sensitivity | Sensitivity | Sensitivity | |
|---|---|---|---|---|
| Combination | for AD | for DLB | for FTD | Specificity |
| Combination 1 | 40.43% | 4.55% | 7.94% | 93.91% |
| Combination 2 | 70.21% | 11.36% | 22.22% | 85.28% |
| Combination 3 | 59.57% | 4.55% | 7.94% | 89.85% |
| Combination 4 | 65.96% | 4.55% | 7.94% | 89.85% |
| Combination 5 | 57.45% | 20.45% | 12.70% | 85.79% |
| Combination 6 | 61.70% | 11.36% | 14.29% | 87.31% |
| Combination 7 | 63.83% | 15.91% | 9.52% | 89.85% |
| Combination 8 | 80.85% | 11.36% | 22.22% | 85.79% |
| Combination 9 | 85.11% | 11.36% | 22.22% | 85.79% |
| Combination 10 | 80.85% | 20.45% | 22.22% | 81.22% |
| Combination 11 | 72.34% | 15.91% | 25.40% | 83.25% |
| Combination 12 | 82.98% | 20.45% | 23.81% | 85.79% |
| Combination 13 | 72.34% | 4.55% | 7.94% | 89.85% |
| Combination 14 | 70.21% | 20.45% | 12.70% | 83.25% |
| Combination 15 | 76.60% | 11.36% | 14.29% | 87.82% |
| Combination 16 | 74.47% | 15.91% | 9.52% | 87.82% |
| Combination 17 | 76.60% | 20.45% | 12.70% | 83.25% |
| Combination 18 | 80.85% | 11.36% | 14.29% | 87.82% |
| Combination 19 | 78.72% | 15.91% | 9.52% | 87.82% |
| Combination 20 | 72.34% | 27.27% | 17.46% | 83.25% |
| Combination 21 | 76.60% | 31.82% | 14.29% | 81.22% |
| Combination 22 | 78.72% | 20.45% | 15.87% | 85.79% |
| Combination 23 | 85.11% | 11.36% | 22.22% | 85.79% |
| Combination 24 | 87.23% | 20.45% | 22.22% | 81.22% |
| Combination 25 | 80.85% | 15.91% | 25.40% | 83.25% |
| Combination 26 | 87.23% | 20.45% | 23.81% | 85.79% |
| Combination 27 | 91.49% | 20.45% | 22.22% | 81.22% |
| Combination 28 | 85.11% | 15.91% | 25.40% | 83.25% |
| Combination 29 | 91.49% | 20.45% | 23.81% | 85.79% |
| Combination 30 | 80.85% | 27.27% | 25.40% | 81.22% |
| Combination 31 | 89.36% | 31.82% | 23.81% | 81.22% |
| Combination 32 | 85.11% | 27.27% | 26.98% | 83.25% |
| Combination 33 | 76.60% | 20.45% | 12.70% | 83.25% |
| Combination 34 | 80.85% | 11.36% | 14.29% | 87.82% |
| Combination 35 | 78.72% | 15.91% | 9.52% | 87.82% |
| Combination 36 | 82.98% | 27.27% | 17.46% | 83.25% |
| Combination 37 | 82.98% | 31.82% | 14.29% | 81.22% |
| Combination 38 | 85.11% | 20.45% | 15.87% | 85.79% |
| Combination 39 | 87.23% | 27.27% | 17.46% | 83.25% |
| Combination 40 | 87.23% | 31.82% | 14.29% | 81.22% |
| Combination 41 | 89.36% | 20.45% | 15.87% | 85.79% |
| Combination 42 | 85.11% | 36.36% | 19.05% | 81.22% |
| Combination 43 | 91.49% | 20.45% | 22.22% | 81.22% |
| Combination 44 | 85.11% | 15.91% | 25.40% | 83.25% |
| Combination 45 | 91.49% | 20.45% | 23.81% | 85.79% |
| Combination 46 | 87.23% | 27.27% | 25.40% | 81.22% |
| Combination 47 | 91.49% | 31.82% | 23.81% | 81.22% |
| Combination 48 | 87.23% | 27.27% | 26.98% | 83.25% |
| Combination 49 | 91.49% | 27.27% | 25.40% | 81.22% |
| Combination 50 | 95.74% | 31.82% | 23.81% | 81.22% |
| Combination 51 | 91.49% | 27.27% | 26.98% | 81.22% |
| Combination 52 | 89.36% | 36.36% | 26.98% | 81.22% |
| Combination 53 | 87.23% | 27.27% | 17.46% | 83.25% |
| Combination 54 | 87.23% | 31.82% | 14.29% | 81.22% |
| Combination 55 | 89.36% | 20.45% | 15.87% | 85.79% |
| Combination 56 | 89.36% | 36.36% | 19.05% | 81.22% |
| Combination 57 | 93.62% | 36.36% | 19.05% | 81.22% |
| Combination 58 | 91.49% | 27.27% | 25.40% | 81.22% |
| Combination 59 | 95.74% | 31.82% | 23.81% | 81.22% |
| Combination 60 | 91.49% | 27.27% | 26.98% | 83.25% |
| Combination 61 | 91.49% | 36.36% | 26.98% | 81.22% |
| Combination 62 | 95.74% | 36.36% | 26.98% | 81.22% |
| Combination 63 | 93.62% | 36.36% | 19.05% | 81.22% |
| Combination 64 | 95.74% | 36.36% | 26.98% | 81.22% |

The results in Table 6 show that the detected specificity of each combination is good (81% or above), and the detected specificity of some combinations reached 85% and even 90% or above. The detected sensitivity for patients with AD was several times higher than that for FTD and DLB. For example, the sensitivity of Combination 13 for AD reached 72.34%, whereas the detected sensitivity thereof for FTD was only 7.94%, and the detected sensitivity for DLB was also very low (only 4.55%), thereby achieving good identifying and distinguishing significance for AD relative to FTD and DLB. During later application(s) and/or development, one or more of the candidate antigens or antigen combinations provided by the present disclosure may be selected to be included in a kit for diagnosing AD and/or identifying AD and distinguishing AD from FTD and DLB.

### Example 5: Display of the Effectiveness of Candidate Antigen Combinations and Corresponding Full-Length Protein Combinations for Detecting AD, FTD and DLB

1. 4 groups of different antigen combinations were selected from Table 5 in Example 4. All of the proteins or protein fragments were as shown in Table 3. The corresponding full-length protein combinations were obtained based on a full-length protein corresponding to each protein fragment in the antigen combinations in Groups 1 to 4, which were labeled as Control Groups 1 to 4 respectively, as shown in Table 7. Where the proteins in Table 3 are already full-length proteins, the proteins were not adjusted.

TABLE 7
List of antigen combinations

| Group | Antigen Combination |
|---|---|
| Group 1 | SERF2, SNAP25, RAGE |
| Group 2 | SERF2, SNAP25, P21, DNAJC8 |
| Group 3 | SERF2, SNAP25, P21, DNAJC8, JMJD2D |
| Group 4 | SERF2, SNAP25, P21, DNAJC8, JMJD2D, |
| | MAPT, ASXL1, RAGE |
| Full-length protein | SERF2, SNAP25, RAGE |
| control group 1 | |
| Full-length protein | SERF2, SNAP25, P21, DNAJC8 |
| control group 2 | |
| Full-length protein | SERF2, SNAP25, P21, DNAJC8, |
| control group 3 | JMJD2D |
| Full-length protein | SERF2, SNAP25, P21, DNAJC8, |
| control group 4 | JMJD2D, MAPT, ASXL1, RAGE |

2. Each sample in Example 4 was tested for sensitivity towards AD, FTD and DLB and specificity for AD using the detection method in Example 2 and each group/antigen combination in Table 7. Results are shown in Table 8.

TABLE 8
Detection results for each Group/antigen combination

| | Sensitivity | Sensitivity | Sensitivity | |
|---|---|---|---|---|
| Combination | for AD | for DLB | for FTD | Specificity |
| Group 1 | 63.83% | 9.52% | 15.91% | 89.85% |
| Group 2 | 72.34% | 7.94% | 4.55% | 89.85% |
| Group 3 | 80.85% | 14.29% | 11.36% | 87.82% |
| Group 4 | 95.74% | 26.98% | 36.36% | 81.22% |
| Control group 1 | 57.45% | 17.46% | 22.73% | 83.25% |
| Control group 2 | 70.21% | 25.40% | 27.27% | 80.20% |
| Control group 3 | 78.72% | 30.16% | 27.27% | 74.62% |
| Control group 4 | 89.36% | 52.38% | 52.27% | 56.85% |

The results in Table 8 show that, when compared with combinations using only full-length proteins, the detection specificity of each combination containing antigen fragments was good (81% or above). Meanwhile, each combination containing antigen fragments had high sensitivity for detecting AD, as well as lower sensitivities for detecting FTD and DLB, thereby achieving improved significance and/or ability to identify AD autoantibodies and distinguish AD autoantibodies from FTD and DLB autoantibodies.

The above-described examples are merely the descriptions of preferred implementations of the present disclosure, and do not limit the scope of the present disclosure. Various modifications, variations, alterations, and substitutions made by those of ordinary skill in the art to the technical solutions of the present disclosure are intended to fall within the scope of protection identified by the claims of the present disclosure without departing from the design and/or spirit of the present disclosure.

What is claimed is:

1. An antigen combination, comprising a SERF2 protein and/or a SNAP25 protein.

2. The antigen combination according to claim 1, comprising the SERF2 protein and the SNAP25 protein.

3. The antigen combination according to claim 2, wherein the SERF2 protein has the amino acid sequence shown in SEQ ID NO:7, and the SNAP25 protein has the amino acid sequence shown in SEQ ID NO:8.

4. The antigen combination according to claim 1, further comprising one or more protein fragments selected from the group consisting of a MAPT protein fragment, a RAGE protein fragment, an ASXL1 protein fragment, a JMJD2D protein fragment, a P21 protein fragment and a DNAJC8 protein fragment.

5. The antigen combination according to claim 2, further comprising one or more protein fragments selected from the group consisting of a MAPT protein fragment, a RAGE protein fragment, an ASXL1 protein fragment, a JMJD2D protein fragment, a P21 protein fragment and a DNAJC8 protein fragment.

6. The antigen combination according to claim 5, wherein the one or more protein fragments include the P21 protein fragment and the DNAJC8 protein fragment.

7. The antigen combination according to claim 6, wherein the P21 protein fragment has the amino acid sequence shown in SEQ ID NO:2, and the DNAJC8 protein fragment has the amino acid sequence shown in SEQ ID NO:3.

8. The antigen combination according to claim 6, wherein the one or more protein fragments further include the JMJD2D protein fragment.

9. The antigen combination according to claim 8, wherein the P21 protein fragment has the amino acid sequence shown in SEQ ID NO:2, the DNAJC8 protein fragment has the amino acid sequence shown in SEQ ID NO:3, and the JMJD2D protein fragment has the amino acid sequence shown in SEQ ID NO:6.

10. The antigen combination according to claim 8, wherein the one or more protein fragments further include the MAPT protein fragment, the ASXL1 protein fragment, and the RAGE protein fragment.

11. The antigen combination according to claim 10, wherein the P21 protein fragment has the amino acid sequence shown in SEQ ID NO:2, the DNAJC8 protein fragment has the amino acid sequence shown in SEQ ID NO:3, the JMJD2D protein fragment has the amino acid sequence shown in SEQ ID NO:6, the MAPT protein fragment has the amino acid sequence shown in SEQ ID NO:1, the RAGE protein fragment has the amino acid sequence shown in SEQ ID NO:4, and the ASXL1 protein fragment has the amino acid sequence shown in SEQ ID NO:5.

12. The antigen combination according to claim 6, wherein the one or more protein fragments further include the RAGE protein fragment.

13. The antigen combination according to claim 10, wherein the P21 protein fragment has the amino acid sequence shown in SEQ ID NO:2, the DNAJC8 protein fragment has the amino acid sequence shown in SEQ ID NO:3, and the RAGE protein fragment has the amino acid sequence shown in SEQ ID NO:4.

14. A kit for detecting and/or identifying Alzheimer's disease, comprising the antigen combination according to claim 1.

15. A kit for detecting and/or identifying Alzheimer's disease, comprising the antigen combination according to claim 2.

16. The kit according to claim 15, wherein the SERF2 protein has the amino acid sequence shown in SEQ ID NO:7, and the SNAP25 protein has the amino acid sequence shown in SEQ ID NO:8.

17. The kit according to claim 16, further comprising a MAPT protein fragment having the amino acid sequence shown in SEQ ID NO:1, a RAGE protein fragment having the amino acid sequence shown in SEQ ID NO:4, an ASXL1 protein fragment having the amino acid sequence shown in SEQ ID NO:5, a JMJD2D protein fragment having the amino acid sequence shown in SEQ ID NO:6, a P21 protein fragment having the amino acid sequence shown in SEQ ID NO:2, and a DNAJC8 protein fragment having the amino acid sequence shown in SEQ ID NO:3.

18. A kit for distinguishing Alzheimer's disease from frontotemporal dementia and dementia with Lewy bodies, comprising the antigen combination according to claim 2.

19. The kit according to claim 18, wherein the SERF2 protein has the amino acid sequence shown in SEQ ID NO:7, and the SNAP25 protein has the amino acid sequence shown in SEQ ID NO:8.

20. The kit according to claim 19, further comprising a MAPT protein fragment having the amino acid sequence shown in SEQ ID NO:1, a RAGE protein fragment having the amino acid sequence shown in SEQ ID NO:4, an ASXL1 protein fragment having the amino acid sequence shown in SEQ ID NO:5, a JMJD2D protein fragment having the amino acid sequence shown in SEQ ID NO:6, a P21 protein fragment having the amino acid sequence shown in SEQ ID NO:2, and a DNAJC8 protein fragment having the amino acid sequence shown in SEQ ID NO:3.


SEQUENCE LISTING (8 sequences)

SEQ ID NO: 1 - protein, 257 aa, Homo sapiens
SGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 163 aa, Homo sapiens
SEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 3 - protein, 140 aa, Homo sapiens
EQKKRALDVIQAGKEYVEHTVKERKKQLKKEGKPTIVEEDDPELFKQAVYKQTMKLFAELEIKRKEREAKEMHERKRQREEEIEAQEKAKREREWQKNFEESRDGRVDSWRNFQANTKGKKEKKNRTFLRPPKVKMEQRE

SEQ ID NO: 4 - protein, 32 aa, Homo sapiens
AQNITARIGEPLVLKCKGAPKKPPQRLEWKLN

SEQ ID NO: 5 - protein, 84 aa, Homo sapiens
MKDKQKKKKERTWAEAARLVLENYSDAPMTPKQILQVIEAEGLKEMRSGTSPLACLNAMLHSNSRGGEGLFYKLPGRISLFTLK

SEQ ID NO: 6 - protein, 160 aa, Homo sapiens
METMKSKANCAQNPNCNIMIFHPTKEEFNDFDKYIAYMESQGAHRAGLAKIIPPKEWKARETYDNISEILIATPLQQVASGRAGVFTQYHKKKKAMTVGEYRHLANSKKYQTPPHQNFEDLERKYWKNRIYNSPIYGADISGSLFDENTKQWNLGHLGTI

SEQ ID NO: 7 - protein, 59 aa, Homo sapiens
MTRGNQRELARQKNMKKQSDSVKGKRRDDGLSAAARKQRDSEIMQQKQKKANEKKEEPK

SEQ ID NO: 8 - protein, 206 aa, Homo sapiens
MAEDADMRNELEEMQRRADQLADESLESTRRMLQLVEESKDAGIRTLVMLDEQGEQLERIEEGMDQINKDMKEAEKNLTDLGKFCGLCVCPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSG